(19) United States
(12) Patent Application Publication
(10) Pub. No.: US 2023/0310090 A1
(43) Pub. Date: Oct. 5, 2023

(54) NONINTRUSIVE TARGET TRACKING METHOD, SURGICAL ROBOT AND SYSTEM

(71) Applicant: Tsinghua University, Beijing (CN)

(72) Inventors: Gangtie Zheng, Beijing (CN); Shijie Zhu, Beijing (CN); Yu Chen, Beijing (CN); Bicong Zhang, Beijing (CN); Yongwei Pan, Beijing (CN); Zhe Zhao, Beijing (CN); Jiuzheng Deng, Beijing (CN)

(21) Appl. No.: 18/128,819

(22) Filed: Mar. 30, 2023

(30) Foreign Application Priority Data
Mar. 30, 2022 (CN) 202210597366.6

(51) Int. Cl. A61B 34/20; A61B 90/00

(52) U.S. Cl. CPC A61B 34/20; A61B 90/36; A61B 2034/2046

(57) ABSTRACT

A target tracking method and system for use with a surgical robot is disclosed. The method includes: acquiring a visible light image and a depth image of a marker attached on a patient's body surface, where the marker is provided with a black and white checkerboard pattern, and a two-dimensional code is arranged inside squares of the checkerboard; performing two-dimensional code detection on the visible light image to obtain the checkerboard corners' 2D coordinates and the IDs of the two-dimensional codes on the marker; and obtaining 3D coordinates of checkerboard corners in the marker by using the depth image, 2D code corners' coordinates and 2D code ID. According to the 3D coordinates of the checkerboard corner, the position information of the tracked target in the 3D space is obtained.

### CROSS-REFERENCE TO RELATED APPLICATIONS

This application claims the benefit of priority under 35 U.S.C. § 119 from Chinese Patent Application No. 202210597366.6, filed on Mar. 30, 2022, the disclosure of which is incorporated herein by reference in its entirety.

### TECHNICAL FIELD

The present invention generally relates to the field of surgical robots for providing assistance to orthopedic surgery, interventional ablation and other surgeries that require accurate tool space motion, tracking, and positioning. More particularly, a method of using a nonintrusive, planer marker that can be arranged on patients' body surfaces is disclosed. Such markers can replace widely used space markers which are intrusive, as they need to be inserted into a patient's skeleton for tracking the motion of a patient's body part and guiding the motion of a surgical tool during surgery.

### BACKGROUND

Generally, in the process of a robot assisted surgery, a robot tracks and positions a specific part of a patient's body, moves surgical tools interacting with the human body part by following a pre-planned target path, and assists surgeons to carry out operations because of its merits of having high accuracy, excellent stability, and reliability. However, due to respiratory movement and the flexibility of a patient's body, as well as motions caused by accidental contact of a patient's body with medical staff surrounding an operating table, the relative position and posture of the patient's body with respect to the robot may change regularly and/or randomly. This in turn influences the positioning and tracking accuracy of surgical tools and can even result in operation failure.

For this reason, some related technologies propose to track the respiratory movement, body movement and posture change of a patient during an operation through a group of certain markers attached to the human body (Sergej Kammerzell, Uwe Bader and Benoit Mollard, Method and apparatus for positioning a one prosthesis using a localization system, U.S. Pat. No. 7,594,933B2, Sep. 29, 2009). Currently, those markers used in orthopedic surgery (Jeremy Weinstein, Andrei Danilchenko, and Jose Luis Moctezuma de la Barrera, Systems and methods for surgical navigation, U.S. Ser. No. 10/499,997B2, Dec. 10, 2019) and neurosurgery (Nahum Bertin and Blondel Lucien, Multi-application robotized platform for neurosurgery and resetting method, U.S. Pat. No. 8,509,503, Aug. 13, 2013) (Luc Gilles Charron, Michael Frank and Gunter Wood, Surgical imaging sensor and display unit, and surgical navigation system associated therewith, U.S. Ser. No. 11/160,614B2, Nov. 2, 2021) have the form of three dimensional structures and thus have to be inserted into a patient's skeleton for a firm and stable connection. Therefore, they generally have a metal pin for being inserted into the human skeleton. This way of arranging markers on patient's body can be called intrusive arrangement, or the marker can be considered as an intrusive marker.

A typical marker has several spaced, distributed optical reflective points, which usually are in the shape of ball. (Nelson L. Groenke and Holger-Claus Rossner, Medical device for surgical navigation system and corresponding method of manufacturing, U.S. Ser. No. 10/537,393B2, Jan. 21, 2020). The implantation process for such markers causes injury to the human body by producing incision and bone damage, resulting in secondary trauma, and even secondary fracture near the bone pin sites after the operation, as warned in MAKOplasty® Partial Knee Application User Guide (206388 Rev 02, p. 69), based on some research results published before (1. C. Li, T. Chen, Y Su, P. Shao, K Lee, and W. Chen, Periprosthetic Femoral Supracondylar Fracture After Total Knee Arthroplasty With Navigation System. The Journal of Arthroplasty 2006; 12:049. 2. Pins. D. Hoke, S. Jafari, F. Orozco, and A. Ong, Tibial Shaft Stress Fractures Resulting from Placement of Navigation Tracker, The Journal of Arthroplasty 2011; 26:3. 3. H. Jung, Y. Jung. K. Song, S. Park, and J. Lee, Fractures Associated with Computer-Navigated Total Knee Arthroplasty. The Journal of Bone and Joint Surgery, [BR] 2007; 89:2280-4. 4. H. Maurer, C. Wimmer, C. Gegenhuber, C. Bach, and M. Krismer, Knee pain caused by a fiducial marker in the medial femoral condyle. M. Nogler, Acta Orthop Scand, 2001; 72 (5):477-480. 5. R. Wysocki, M. Sheinkop, W. Virkus, and C. Della Valle, Femoral Fracture Through a Previous Pin Site After Computer-Assisted Total Knee Arthroplasty, The Journal of Arthroplasty 2007; 03:019). Such injuries belong to the category of iatrogenic harm and should be totally avoided.

To solve this problem, an alternative approach is needed.

### SUMMARY

The present invention aims to solve the problem of iatrogenic harm caused by using intrusive markers for tracking the motion of a target motion and guiding the motion of a medical tool. Therefore, an object of the present invention is to propose a target tracking method, through which a marker does not need to be placed inside a patient's body, and in the meantime to ensure a patient's safety and achieve high motion tracking accuracy. The second object of the present invention is to propose a surgical robot. The third object of the present invention is to develop a target tracking system.

An embodiment of tracking an area on a patient's body, which is taken as a target, i.e., the target tracking method, is to use planer markers to replace prior space markers. Such a planer marker, which can be either flexible or rigid, is provided with a black-and-white checkerboard pattern, and white checkerboard portions are internally provided with a two-dimensional code or figure, which are all called codes for simplifying the description in the following description. Such a marker can be directly arranged on patient body surface by medical transparent tape, or medical transparent film, or simply glue. Therefore, such a marker can be called a nonintrusive marker. The method for its application in tracking a target includes: obtaining the visible light image and depth image of the marker; performing two-dimensional code detection on the visible light image to obtain 2D coordinates of two-dimensional code corners and the identifiers (IDs) of the two-dimensional codes in the marker; according to the depth image, the 2D coordinates of the 2D code corners and the IDs of the 2D codes, 3D coordinates of the checkerboard corners in the marker are obtained; with the 3D coordinates of the checkerboard corners, the position information of the tracked target in 3D space is obtained, wherein the position information is used to track the target.

To conduct a surgery with the above planer markers, said nonintrusive marker, the second aspect of the embodiment of the invention is a robot, which comprises: a visible image acquisition module for acquiring the visible image of the marker attached to the surface of the tracked target; a depth image acquisition module for acquiring a depth image of the marker; an image processing module for processing image information. To track and guide the motion and attitude of the robot, a soft planer marker as mentioned above is arranged on a last section of the robot that connects medical tools. An execution module for generating a motion command of the robot according to the continuously obtained position information of the tracked target and also the robot in the 3D space, and controlling the robot to follow the tracked target and planned motion path in the 3D space, is also provided. The execution module can move a robotic arm or portion thereof, and can move a surgical tool attached to the arm or otherwise mechanically connected to the surgical robot. Surgical tools that can be used include those known to the art, such as drill guides, drills, puncture needles, scissors, graspers, and needle holders. The execution module can further use such surgical tools to perform a surgical operation with the surgical robot, such as a drilling operation or a cutting operation.

The third aspect of the embodiment of the invention proposes a target tracking system. Such a system includes the nonintrusive marker as described according the embodiment of the first aspect of the present invention and a robot according to the embodiment of the second aspect of the present invention.

According to the target tracking method, robot, and system of the invention, by attaching nonintrusive markers on the surface of a patient's body, i.e., the tracked target, the occurrence of iatrogenic harms caused by the intrusion of intrusive markers into the interior of the patient's body as in prior technologies can be avoided while still providing tracking and navigation accuracy. Additional aspects and advantages of the invention will be given in the following description, and some will become apparent from the following description, or will be known through the practice of the invention.

### BRIEF DESCRIPTION OF THE DRAWINGS

Together with the specification, these drawings illustrate exemplary embodiments of the present invention, and, together with the description, are used to explain the principles and implementing procedures of the present invention.

FIG. 1 is a flowchart of a target tracking method according to an embodiment of the present invention;

FIG. 2 is a schematic diagram of the nonintrusive marker of the first example of the present invention;

FIG. 3 is a schematic diagram of the nonintrusive marker of the second example of the present invention;

FIG. 4 is a schematic diagram of the nonintrusive marker of the third example of the present invention;

FIG. 5 is a schematic diagram of a single two-dimensional code template of an example of the present invention;

FIG. 6 is a flowchart of step S102 of a target tracking method according to an embodiment of the present invention;

FIG. 7 is a schematic diagram of matching a two-dimensional code template with a visible light image in an example of the present invention;

FIG. 8 is a schematic flowchart of a target tracking method according to another embodiment of the present invention;

FIG. 9 is a flowchart of step S103 of a target tracking method according to an embodiment of the present invention;

FIG. 10 is a flowchart of an example of the present invention for obtaining a key area of interest in a visible light image according to the 2D coordinates of a 2D code corner and the 2D code ID;

FIG. 11 is a schematic diagram of a homography transformation from a standard image of a marker to a visible light image of an example of the present invention;

FIG. 12(a) is a schematic diagram of the position of the tracked target in the visible light image according to an example of the present invention;

FIG. 12(b) is a schematic diagram of the position of the tracked target in the depth image of an example of the present invention;

FIG. 12(c) is the present invention A schematic diagram of the position of an example tracked target in 3D space;

FIG. 13 is a schematic structural diagram of a robot according to an embodiment of the present invention;

FIG. 14 is a schematic structural diagram of a target tracking system according to an embodiment of the present invention.

### DETAILED DESCRIPTION

Embodiments of the present invention are described in detail below, examples of which are shown in the accompanying drawings, wherein the same or similar reference numbers throughout represent the same or similar elements or elements having the same or similar functions. The embodiments described below by reference to the accompanying drawings are exemplary and are intended to explain the present invention, but should not be understood as limiting the present invention.

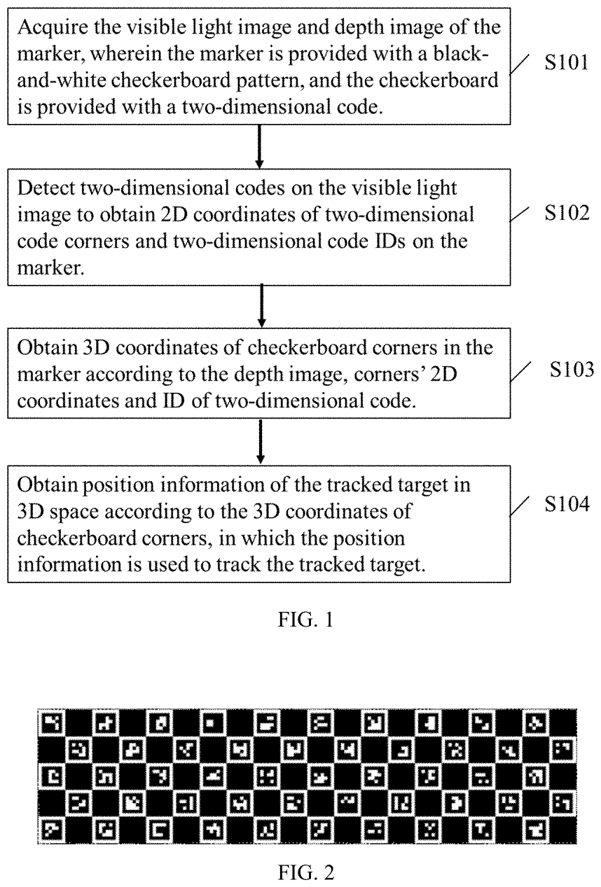

The embodiment of the target tracking method, robot and system of the present invention are described below with reference to FIGS. 1-14 and specific embodiments.

FIG. 1 is a flowchart of a target tracking method according to an embodiment of the present invention. As shown in FIG. 1, the target tracking method provided by this embodiment includes the following steps:

In step S101, a visible light image and depth image of the marker attached to the surface of the tracked target is obtained, wherein the marker is provided with a black-and-white checkerboard pattern, and the white checkerboard squares are provided with a two-dimensional code. Preferably, each of the white squares comprises a two-dimensional code. As used herein, “checkerboard” refers to a regular pattern of squares of alternating colors in the manner typically provided on a checkerboard, and the colors used typically are black and white. It should be understood that the use of black and white as colors is arbitrary, and that “black” and “white” squares can refer to areas of any of two contrasting colors.

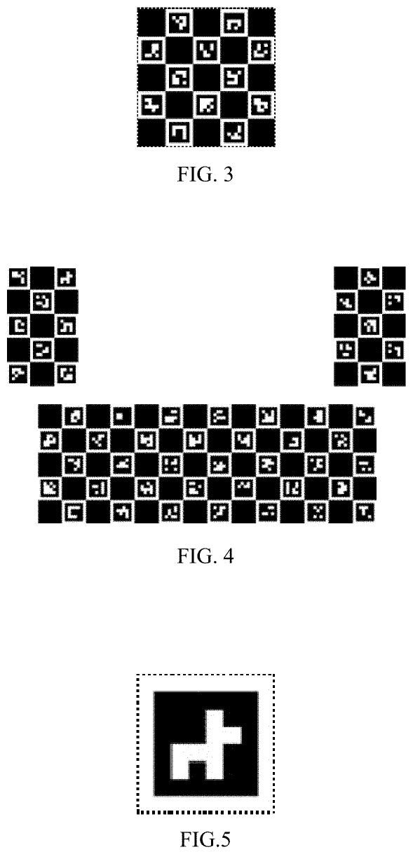

In some embodiments, the marker can be formed from a flexible planar base and can be cut into any shape. The marker is provided with a black-and-white checkerboard pattern, and a two-dimensional code/figure is arranged inside the white checkerboard squares, as shown in FIG. 2 and FIG. 3. Further, in some examples, in order to ensure the best view in the tracking process, as shown in FIG. 4, the checkerboard with two-dimensional code/figure can be cut into any shape, and the checkerboard can be combined in any way according to actual requirements to obtain the checkerboard pattern on the final marker.

It should be noted that in the checkerboard patterns as shown in FIG. 2, FIG. 3 and FIG. 4, the two-dimensional code/figure is stored only in the white checkerboard (half of the checkerboard). This design can ensure the detection rate of the two-dimensional code/figure during subsequent two-dimensional code/figure detection, and will not be interfered by adjacent two-dimensional codes/figures. Moreover, as shown in FIG. 5, the two-dimensional codes inside each white checkerboard in the checkerboard pattern are preferably unique and directional in the checkerboard used in a surgical procedure. When designing the marker, it is necessary to ensure that there is dissimilarity between different two-dimensional codes, so as to reduce the probability of identification errors or having one two-dimensional code being wrongly identified as other two-dimensional codes/figures in the process of two-dimensional code/figure detection.

As an example, in practical application, the required number of two-dimensional codes can be calculated according to an actual application scenario. Generally, a long bar shape is often used for the scenario that needs cut and/or combination, for example, 5 mm×20 mm. For those scenarios that do not need to be cut and combined, a square shape is often used, such as 5 mm×5 mm. It should be noted that the above design method is only exemplary and does not serve as a limitation on the embodiments of the present invention.

In step S102, two-dimensional code detection is carried out on the visible light image to obtain 2D coordinates of two-dimensional code corners and two-dimensional code IDs on the marker.

As a feasible implementation, when detecting the two-dimensional code of the visible light image, a corresponding number of information dictionaries can be generated in advance according to the number of two-dimensional codes selected. Each information dictionary corresponds to a two-dimensional code containing information and its corresponding ID. When detecting the two-dimensional code of the visible light image later, after detecting the two-dimensional code, the 2D coordinates of the corners of the two-dimensional code and the ID of the information dictionary generated by the two-dimensional code can be obtained.

It should be noted that when detecting two-dimensional codes for visible light images, at least two noncollinear two-dimensional codes need to be detected. Each two-dimensional code detection includes four two-dimensional code corners. Through the two two-dimensional codes, the global (i.e., on the marker) other two-dimensional codes can be inferred.

In step S103, according to the depth image, 2D coordinates of two-dimensional code corners and 2D code ID, the 3D coordinates of checkerboard corners in the marker are obtained.

In step S104, according to the 3D coordinates of checkerboard corners, the position information of the tracked target in 3D space is obtained, in which the position information is used to track the tracked target.

Specifically, after obtaining the 3D coordinates of the checkerboard corners on the marker in step S103, the position information of the tracked target, on which the marker is arranged, in the 3D space can be obtained, and the target tracking can be realized by continuously obtaining the 3D coordinates of the marker in the 3D space from continuously taken images.

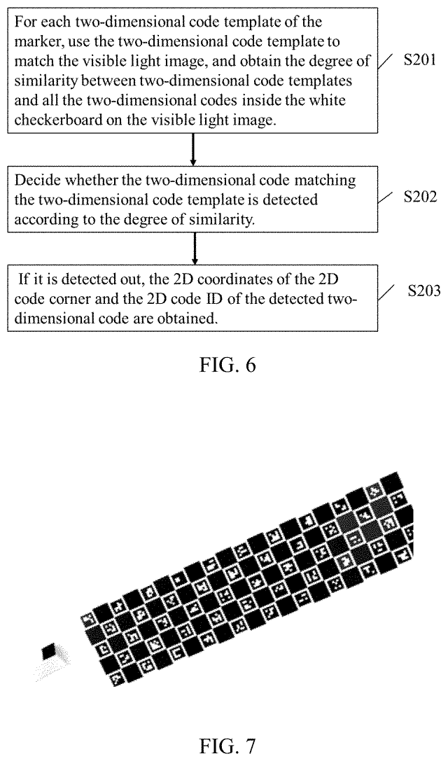

As a possible implementation, as shown in FIG. 6, in the target tracking method embodiment of the present invention, two-dimensional code detection is performed on the visible light image to obtain the two-dimensional code corner's 2D coordinates and two-dimensional code ID on the marker, which may include the following steps.

In step S201, for each two-dimensional code template of the marker, use the two-dimensional code template to match the visible light image, and obtain the degree of similarity between two-dimensional code templates and all the two-dimensional codes inside the white checkerboard on the visible light image.

For example, in some embodiments, the visible light image of the marker can be scaled at various scales first, and then the template for each two-dimensional code can be used to match the visible light image, as shown in FIG. 7. During the matching process, the degree of similarity between the two-dimensional code template and the white checkerboard on the marker in the scaled visible light image can be obtained through an image recognition algorithm.

In step S202, it is determined whether the two-dimensional code matching the two-dimensional code template is detected according to the degree of similarity.

It should be noted that only when the degree of similarity is higher than a preset (predetermined) threshold, the detection of two-dimensional code, which matches to a two-dimensional code template, can be considered as successful. The threshold can be set according to the actual situation, such as 95%.

In step S203, if it is detected out, the 2D coordinates of the 2D code corner and the 2D code ID of the detected two-dimensional code are obtained.

Specifically, as proposed in the above embodiment, each two-dimensional code has its corresponding information dictionary, and the information dictionary includes the two-dimensional code IDs. Therefore, when the two-dimensional code is detected, the two-dimensional code ID of the detected two-dimensional code can be obtained by retrieving the relevant data in its information dictionary.

Thus, through steps S201-S203, the corner 2D coordinates and ID of the two-dimensional code detected in the visible light image on the marker can be obtained.

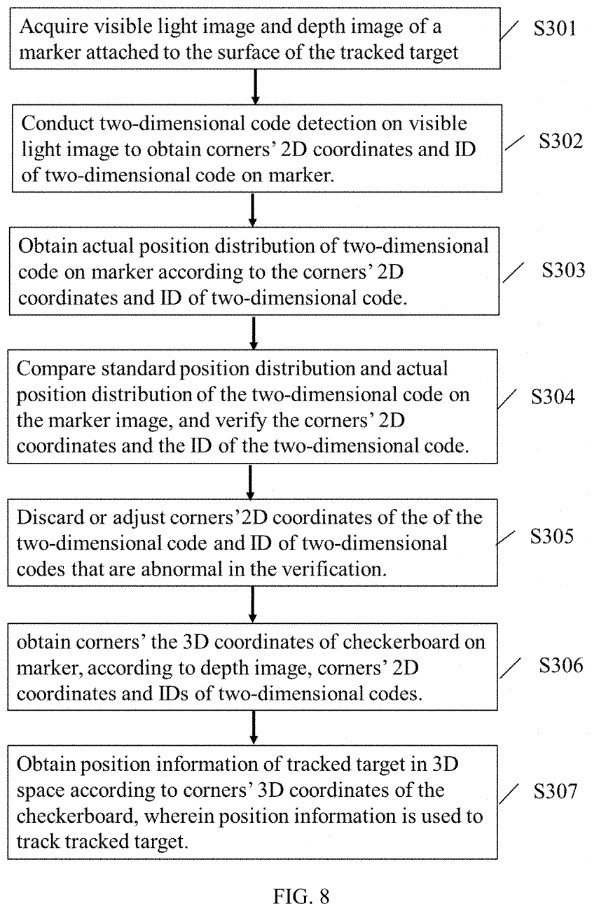

Further, in some embodiments of the invention, in order to ensure the stability of the working process of the target tracking method, it may also necessary to verify the 2D coordinates of the two-dimensional code corners and the two-dimensional code ID of the obtained markers. FIG. 8 is a schematic flowchart of the target tracking method of another embodiment of the invention, as shown in FIG. 8. The target tracking method may include the following steps:

Step S301: acquiring a visible light image and a depth image of a marker attached to the surface of the tracked target, wherein the marker is provided with a black and white checkerboard pattern, and a two-dimensional code is provided inside the white checkerboard.

Step S302: conducting two-dimensional code detection on the visible light image to obtain corners' 2D coordinates and ID of the two-dimensional code on the marker.

Step S303: according to the 2D coordinates of the corners of the two-dimensional code and the two-dimensional code ID, the actual position distribution of the two-dimensional code on the marker is obtained.

Step S304: comparing the standard position distribution and the actual position distribution of the two-dimensional code on the marker image, and verify the corners' 2D coordinates and the ID of the two-dimensional code, respectively.

Step S305: discarding or adjusting the corners' 2D coordinates of the two-dimensional codes and the two-dimensional code IDs that are abnormal in the verification. Abnormal two-dimensional codes and two-dimensional code IDs can be those whose corners' 2D coordinates deviate by more than a predetermined amount. Specifically, in this embodiment, the two-dimensional code detection is performed on the visible light image of the marker. When there is a verification exception in the verification result, the 2D coordinates of the two-dimensional code corner and the two-dimensional code ID with the verification exception can be directly discarded. In some embodiments, if there are too many verification exceptions in the verification results, it may be necessary to feed back the verification situation in time and report the poor quality of the obtained visible light image. In practical applications, such situations may be affected by external illumination (for example, the light changes, the reflection angle changes), occlusion and other problems. At this time, it can be adjusted by external intervention, for example, re-acquiring the visible light image of the marker, so as to ensure the stability and reliability of the subsequent target tracking work.

Step S306: according to the depth image, the 2D coordinates of the corners of the two-dimensional code, and the ID of the two-dimensional code, obtaining the 3D coordinates of the corners of the checkerboard on the marker.

Step S307: obtaining the position information of the tracked target in the 3D space according to the 3D coordinates of the corners of the checkerboard, wherein the position information is used to track the tracked target.

It should be noted that the specific implementation method of steps S301, S302, S306 and S307 in this embodiment can refer to the specific implementation process of S101-S104 in the above embodiment of the invention, and hence will not be repeated here.

In this embodiment, by verifying the corners' 2D coordinates and the ID of the two-dimensional code in the marker, the result of the two-dimensional code detection will be used as a criterion for the stability of the target tracking process. When there are too many abnormal conditions, it can be adjusted with the help of external interventions through timely feedback, so as to improve the reliability of the subsequent target tracking work.

Further, after the 2D coordinates of the corner of the two-dimensional code and the ID of the two-dimensional code have been obtained from the marker, the verification of the corners' 2D coordinates and the ID of the two-dimensional code is completed, and also the correct calibration is obtained, the 3D coordinates of the corners of the checkerboard on the marker can be calculated according to the 2D coordinates and ID of the corners of the normally verified two-dimensional code and the acquired depth image of the marker.

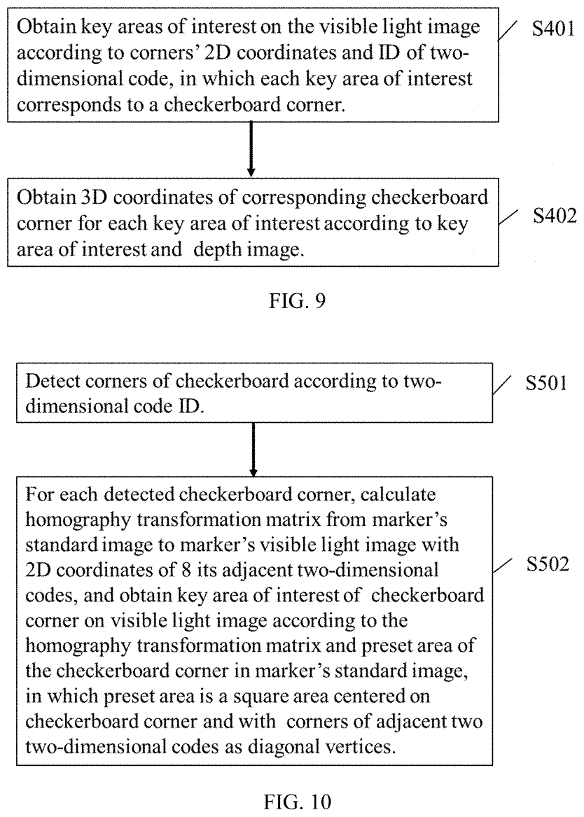

As a possible implementation, as shown in FIG. 9, in the target tracking method of the embodiment of the present invention, the checkerboard corners' coordinates on the marker are obtained according to the depth image, the corners' 2D coordinates of the of the two-dimensional code, and the ID of the two-dimensional code. The calculation process can include the following steps:

Step S401: according to the corners' 2D coordinates and the ID of the two-dimensional code, the key areas of interest in the visible light image are obtained, in which each key area of interest corresponds to a checkerboard corner.

Step S402: for each key area of interest, obtaining the 3D coordinates of the corresponding checkerboard corner according to the key area of interest and the depth image.

In this implementation mode, as an example, as shown in FIG. 10, according to the corner's 2D coordinates and ID of the Two-dimensional code, the key area of interest in the visible light image can be obtained, which can include the following steps:

Step S501: detecting the corners of the checkerboard according to the two-dimensional code ID.

Step S502, for each checkerboard corner detected, calculating the homography transformation matrix from the standard image of the marker to the visible light image of the marker by using the 2D coordinates of 8 its adjacent two-dimensional codes, and obtaining the key area of interest of the checkerboard corner in the visible light image according to the homography transformation matrix and the preset area of the checkerboard corner in the standard image of the marker, in which the preset area is a square area centered on the checkerboard corner and with the corners of adjacent two two-dimensional codes as diagonal vertices.

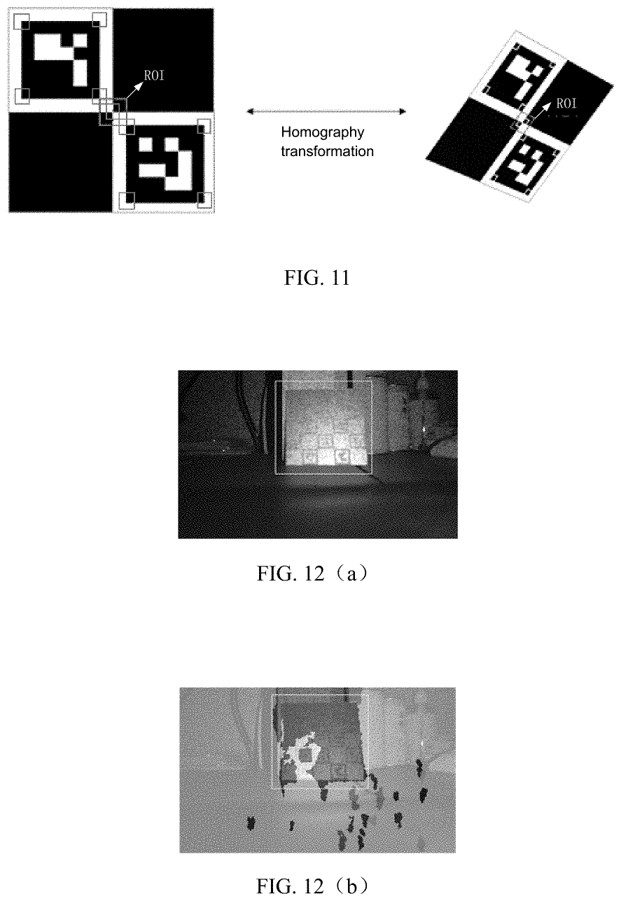

Specifically, FIG. 11 is a schematic diagram of homography transformation from a standard image of a marker to a visible light image of an example of the present invention, in which each checkerboard corner has two adjacent two-dimensional codes in addition to itself, and each two-dimensional code has four corners. In this embodiment, the homography transformation matrix from the standard image of the marker to the visible light image can be established by using the 2D coordinates corresponding to the 8 corners of the two adjacent two-dimensional codes of each detected checkerboard corner. The preset area in the standard image is a square area with the detected checkerboard corner as the center and determined diagonally by the corners of the two adjacent two-dimensional codes. After the preset area is determined, according to the preset region and homography transformation, the key region of interest of the checkerboard corner in the visible image can be obtained. Refer to the ROI (region of interest) section shown in FIG. 11.

Further, as a possible implementation method, after obtaining the key area of interest of the checker angle in the visible light image, the corresponding 3D coordinates of the checker corner can be obtained according to the key area of interest and the depth image. The implementation method can include the following.

As an example, the 3D coordinate of every pixel in a key region of interest is calculated with the following formula:

(x_{3d}ⁱ,y_{3d}ⁱ,z_{3d}ⁱ)=ƒ(x_{depth}ⁱ,y_{depth}ⁱ,x_{2d}ⁱ,y_{2d}ⁱ),

where i∈(1, . . . , N) indicates i-th pixel among N pixels in the key region of interest, (x_{3d}ⁱ, y_{3d}ⁱ, z_{3d}ⁱ) is the 3D coordinate of i-th pixel, (x_{depth}ⁱ, y_{depth}ⁱ) is the 2D coordinate of i-th pixel in the depth image, (x_{2d}ⁱ, y_{2d}ⁱ) is the 2D coordinate of i-th pixel in the visible light image, ƒ is decided by those parameters of the depth image camera and the visible light camera applied.

As an example, the 3D coordinate of corner of the checkerboard is calculated with the following formula:

$$( {x}_{3 d}^{c} , {y}_{3 d}^{c} , {z}_{3 d}^{c} ) = \frac{1}{N} {\sum }_{1}^{N} ( {x}_{3 d}^{i} , {y}_{3 d}^{i} , {z}_{3 d}^{i} ) ,$$

where (x_{3d}^{c}, y_{3d}^{c}, z_{3d}^{c}) denotes the 3D coordinate of checkerboard's corner.

That is to say, in this implementation, the 3D coordinate of the i-th pixel in the area of focus can be calculated firstly according to the 2D coordinate of the i-th pixel in the depth image and the 2D coordinate in the visible light image. After obtaining the 3D coordinate of each pixel in the area of focus, the 3D coordinates (accurate coordinates) of checkerboard's corners can be obtained by weighted averaging of the 3D coordinates of the pixels in the whole area of focus. Subsequently, the method proposed in step S104 of the embodiment of the present invention can be continued, and the position information of the tracked target in 3D space can be obtained according to the obtained 3D coordinates to realize target tracking.

As another possible implementation method, after obtaining the checkerboard's key area of interest on the visible light image, the corresponding 3D coordinates of the checkerboard's corners with angles and/or deformation on the depth image can be obtained according to the key area of interest and the depth image, including the following calculation steps.

The 3D coordinates of four corners of the area of focus are calculated by the following formula:

(x_{3d}ⁱ,y_{3d}ⁱ,z_{3d}ⁱ)=ƒ(x_{depth}ⁱ,y_{depth}ⁱ,x_{2d}ⁱ,y_{2d}ⁱ),

where i∈(1, . . . , 4) indicates the four corners of the area of focus, (x_{3d}ⁱ, y_{3d}ⁱ, z_{3d}ⁱ) is the 3D coordinate of i-th area corner, (x_{depth}ⁱ, y_{depth}ⁱ) denotes the 2D coordinate of i-th area corner in the depth image, (x_{2d}ⁱ, y_{2d}ⁱ) denotes the 2D coordinate of i-th area corner in the visible light image, ƒ is decided by those parameters of the depth image camera and the visible light camera applied.

The center point's coordinates can be obtained by the interpolation of the 3D coordinates of four area corners:

$$( {x}_{3 d}^{c} , {y}_{3 d}^{c} ) = \frac{1}{4} {\sum }_{1}^{4} ( {x}_{3 d}^{i} , {y}_{3 d}^{i} ) .$$

By fitting plane P: k·x+1·y+m·z=0 to make the following formula establish:

(k,l,m)˜argmin Σ₁^{N}(k·x−x_{3d}ⁱ)²+(l·y−y_{3d}ⁱ)²+(m·z−z_{3d}ⁱ)²;

According to x_{3d}^{c}, y_{3d}^{c}, k, l, m and plane formula P, the 3D checkerboard's corner (x_{3d}^{c}, y_{3d}^{c}, z_{3d}^{c}) can be obtained.

That is to say, in this way of implementation, the 3D coordinates of the corner of the i-th area in the focus area can firstly be calculated according to the 2D coordinates of the corner of the i-th area in the depth image and in the visible light image, respectively. After obtaining the 3D coordinates of the corners of the four areas, in the three-dimensional space coordinate system, the plane fitting can be performed according to the 3D coordinates of the corners of the entire key area of interest. At the same time, the 3D coordinates of the corners of the four areas can be obtained on the fitted plane. The coordinate of the center point is obtained by coordinate interpolation, and the 3D coordinates of the corners of the checkerboard are finally obtained according to the obtained coordinates of the center point and the fitted plane.

Optionally, in this embodiment, the camera selects the depth camera. In this implementation, in order to ensure the accuracy of the acquired 3D coordinates of the corners of the checkerboard, the plane fitting can be used to avoid errors that occur at area points or image edges of an image taken by the depth camera.

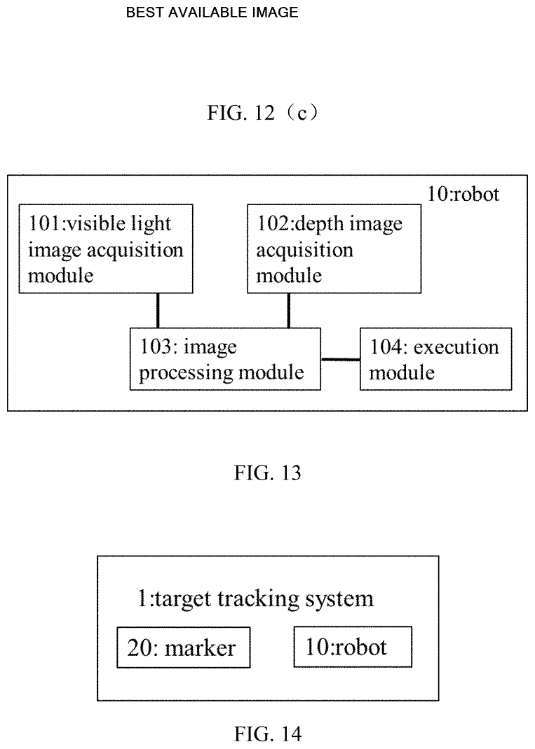

As an example, the 3D coordinates of the corners of the checkerboard are obtained according to the above embodiment. The changes of 3D coordinate of the checkerboard in these consecutive frames are also obtained through the transformation relationship between the corners in the consecutive frame images. That is the 3D coordinate of the tracked target on which the marker is attached is obtained, and thus the target tracking is realized. FIG. 12 is a schematic diagram of the position of the tracked target according to an example of the present invention. FIG. 12(a) is a schematic diagram of the position of the tracked target in the visible light image, 12(b) is a schematic diagram of the position of the tracked target in the depth image, 12(c) is a schematic diagram of the position of the tracked target in 3D space. The tracked target can be obtained according to the transformation relationship among FIG. 12(a), FIG. 12(b) and FIG. 12(c).

It should be noted that, since the two-dimensional code in the marker in the embodiment of the present invention should not only be quickly detected, but also contain enough data position information, the checkerboard corner of the marker can be calculated through subsequent work. 3D coordinates, for example, in a 3D coordinate system, the marker may contain at least 6 degrees of freedom, including 3 translational degrees of freedom and 3 rotational degrees of freedom.

In summary, the target tracking method of the embodiment of the invention, by attaching a black-and-white checkerboard pattern marker on the surface of the tracked target, the problem of invading the marker into the interior of the tracked target in related technologies, which result in iatrogenic injuries, can be avoid. At the same time, when tracking the target, the 2D coordinates and ID of each two-dimensional code corner on the marker can be firstly obtained through two-dimensional code detection, and then they are verified. Only those two-dimensional codes with normal verification results can participate in the subsequent target tracking work, which can greatly improve the stability and reliability of the target tracking process. In the same time, in the process of obtaining the 3D coordinates of the checkerboard corners on the marker, the 3D coordinates of the tracked target on which the marker is attached can be determined by obtaining the transformation relationship between the corners in consecutive frame image. The position of the tracked target and the relationship between the tracked target and its changes along with time can be obtained in real time to ensure the real-time performance of the target tracking process, and since what are achieved are the 3D coordinates of the checkerboard corners, the tracking accuracy can be guaranteed.

Furthermore, the embodiment of the invention proposes a robot 10, as shown in FIG. 13. The robot 10 includes a visible light image acquisition module 101, a depth image acquisition module 102, an image processing module 103 and an execution module 104. Surgical robots which make use of such modules are known to the art, such as those of U.S. Patent Publication No. 20220031398, U.S. Patent Publication No. 20210128261, and U.S. Patent Publication No. 20190125461.

Such robots can be used to perform the method described above, and can include for example a visible light image acquisition module 101 used to obtain the visible light image of the marker attached on the surface of the tracked target, wherein the marker is provided with a black-and-white checkerboard pattern, and the white checkerboard is internally provided with a two-dimensional code. The robot can further include a depth image acquisition module 102 used to acquire the depth image of the marker; an image processing module 103 used to detect the two-dimensional code on the visible light image, obtain the two-dimensional code corners' 2D coordinates and two-dimensional codes' ID on the marker, and obtain the checkerboard corners' 3D coordinates on the marker according to the depth image and 2D coordinates and ID of two-dimensional codes, as well as obtain the position information of the tracked target in the 3D space according to the checkerboard corners' 3D coordinates, wherein the position information is used to track the tracked target; and an execution module 104 used to generate motion instructions to the robot according to the continuously obtained position information of the tracked target in 3D space, and control the robot to follow the tracked target in 3D space. In addition, it should be noted that other compositions and functions of the robot 10 of this embodiment are known to those skilled in the art.

Further, the embodiment of the invention also proposes a target tracking system, as shown in FIG. 14. The target tracking system 1 includes a marker 20 as described herein and a robot 10. The marker 20 is attached on the surface of the object being tracked, wherein the marker 20 can be provided with a black and white checkerboard pattern, with two-dimensional codes arranged inside the white squares of the checkerboard.

It should be noted that, for other specific implementations of the target tracking system in the embodiment of the present invention, reference may be made to the specific implementation of the target tracking method in the above-mentioned embodiment of the present invention.

It should be noted that the logic and/or steps represented in the flowchart or otherwise described herein, for example, can be considered as a sequenced list of executable instructions for realizing logical functions, which can be specifically implemented in any computer-readable medium for the instruction execution system, apparatus, or devices (such as a computer-based system including a processor, or other system that can fetch and execute instructions from an instruction execution system, apparatus, or device), or be used in combination with these instruction execution system, apparatus, or device. For the purposes of this specification, a “computer-readable medium” can be any device that can contain, store, communicate, propagate or transmit programs for use by or in combination with an instruction execution system, apparatus, or device. More specific examples of computer-readable media (non-exhaustive list) include the following: an electrical connection unit (electronic device) with one or more wiring, a portable computer case (magnetic device), a random access memory (RAM), a read-only memory (ROM), an erasable editable read-only memory (EPROM or flash memory), an optical fiber device, and a portable optical disk read-only memory (CDROM). In addition, the computer-readable medium may even be paper or other suitable medium on which the program can be printed, because the program can be obtained electronically, for example, by optical scanning of the paper or other medium, followed by editing, interpretation, or other suitable processing if necessary, and then stored in the computer memory.

It should be understood that various parts of the present invention may be implemented in hardware, software, firmware or a combination thereof. In the above-described embodiments, various steps or methods may be implemented in software or firmware stored in memory and executed by a suitable instruction execution system. For example, if implemented in hardware, as in another embodiment, it can be implemented by any one or a combination of the following techniques known in the art: discrete logic circuits, Application Specific Integrated Circuits (ASICs) with suitable combinational logic gates, Programmable Gate Arrays (PGAs), Field Programmable Gate Arrays (FPGAs), and etc.

In the description of this specification, description with reference to the terms ‘one embodiment,’ ‘some embodiments,’ ‘example,’ ‘specific example,’ or ‘some examples’, etc., mean specific features described in connection with the embodiment or example, structure, material or feature are included in at least one embodiment or example of the present invention. In this specification, schematic expressions of the above terms do not necessarily refer to the same embodiment or example. Furthermore, the particular features, structures, materials or characteristics described may be combined in any suitable manner in any one or more embodiments or examples.

In the description of the invention, it should be understood that orientations or positional relationships indicated by such terms as ‘center’, ‘longitudinal’, ‘transverse’, ‘length’, ‘width’, ‘thickness’, ‘upper’, ‘lower’, ‘front’, ‘rear’, ‘left’, ‘right’, ‘vertical’, ‘horizontal’, ‘top’, ‘bottom’, ‘inner’, ‘outer’, ‘clockwise’, ‘counterclockwise’, ‘axial’, ‘radial’, and etc. are those shown in the attached drawings, which is only for the convenience of describing the invention and simplifying the description, rather than indicating or implying that the device or element must have a specific azimuth, position, be constructed and operated in a specific azimuth, so it cannot be understood as a limitation of the present invention.

In addition, the terms ‘first’ and ‘second’ are only used for descriptive purposes and cannot be understood as indicating or implying relative importance or implicitly indicating the number of indicated technical features. Thus, the features defined with ‘first’ and ‘second’ may explicitly or implicitly include at least one of the features. In the description of the invention, ‘multiple’ means at least two, such as two, three, etc., unless otherwise expressly and specifically defined.

In the present invention, unless otherwise expressly specified and limited, the terms ‘installation/installed’, ‘connection/connected’, ‘fixation/fixed’ and other terms should be understood in a broad sense, for example, they can be fixed connections, detachable connections, integrated, mechanical connection or electrical connection, directly connected or indirectly connected through an intermediate medium, connection within two elements or the interaction relationship between two elements, unless otherwise expressly limited. For those skilled in the art, the specific meaning of the above terms in the invention can be understood according to the specific situation.

In the present invention, unless otherwise expressly specified and limited, the first feature ‘above/on’ or ‘below/under’ the second feature may be in direct contact with the first and second features, or the first and second features may be in indirect contact through an intermediate medium. Moreover, the first feature is ‘above’, ‘on’ and ‘over’ the second feature, but the first feature is directly above or diagonally above the second feature, or it only means that the horizontal height of the first feature is higher than the second feature. The first feature is ‘below’, ‘under’ and ‘beneath’ of the second feature, which can mean that the first feature is directly below or obliquely below the second feature, or simply that the horizontal height of the first feature is less than that of the second feature.

Although the embodiments of the invention have been shown and described above, it can be understood that the above embodiments are exemplary and cannot be understood as limitations of the invention. Those skilled in the art can change, modify, replace and modify the above embodiments within the scope of the invention. All patents, patent publications, and other publications referred to herein are incorporated by reference in their entireties.

What is claimed is:

1. A target tracking method for a surgical robot, wherein the method comprises:
obtaining a visible light image and a depth image of a marker attached on the surface of a tracked target comprising a patient body, wherein the marker is provided with a checkerboard pattern formed by adjacent square-shaped areas having a first or second contrasting color on an upper surface of the marker, wherein each of the square-shaped areas of one of the contrasting colors includes a two-dimensional code having a pattern, and wherein each of the two-dimensional codes is arranged inside one of the square-shaped areas of the checkerboard;
carrying out two-dimensional code detection on the visible light image, and obtaining two-dimensional (2D) coordinates of the corners of the square-shaped areas containing the two-dimensional codes and identifiers (IDs) of each of the two-dimensional codes on the marker;
according to the depth image, the 2D coordinates of the corners of the square-shaped areas containing the two-dimensional codes, and the IDs of the two-dimensional codes, obtaining three-dimensional (3D) coordinates of the corners of the square-shaped areas on the marker;
according to the 3D coordinates of the corners of the square-shaped areas, obtaining position information of the tracked target in 3D space; and
providing the position information to a surgical robot, wherein the position information is used to track the tracked target during a surgical procedure.

2. The target tracking method according to claim 1, wherein the two-dimensional code detection comprises:
for each two-dimensional code template of the marker, matching the two-dimensional code template to the visible light image, wherein the similarity between the two-dimensional code templates and the two-dimensional codes on the checkerboard in the visible light image is obtained;
determining whether a two-dimensional code of a selected square-shaped area matching a two-dimensional code template is detected according to the similarity, wherein if a two-dimensional code matching a corresponding two-dimensional code template is detected according to the similarity, the 2D coordinates of the corners of the selected square-shaped area and the ID of the detected two-dimensional code are obtained.

3. The target tracking method according to claim 1, wherein before obtaining the 3D coordinates of the corners on the marker according to the depth image, the 2D coordinates of the corners of the square-shaped areas containing the two-dimensional codes and the IDs of the two-dimensional codes, the method further comprises:
obtaining an actual position distribution of the two-dimensional codes on the marker according to the 2D coordinates of the corners of the square-shaped areas containing the two-dimensional codes and the IDs of the two-dimensional codes;
comparing a standard position distribution and the actual position distribution of the two-dimensional codes on the marker, and verifying the 2D coordinates of the corners of the square-shaped areas containing the two-dimensional codes and the IDs of the two-dimensional codes;
discarding or adjusting the 2D coordinates of the corners of the square-shaped areas containing the two-dimensional codes and the IDs of the two-dimensional codes that are abnormal in the verification process.

4. The target tracking method according to claim 1, wherein:
according to the 2D coordinates of the corners of the square-shaped areas containing the two-dimensional codes and the IDs of the two-dimensional codes, key areas of interest in the visible light image are obtained, wherein each key area of interest corresponds to a checkerboard corner;
for each key area of interest, the 3D coordinates of the corresponding checkerboard corner are obtained according to the key area of interest and the depth image.

5. The target tracking method according to claim 4, further comprising:
performing checkerboard corner detection according to the two-dimensional code IDs;
for each checkerboard corner detected, calculating a homography transformation matrix from the standard image of the marker to the visible light image of the marker by using the 2D coordinates of eight two-dimensional code corners of two adjacent two-dimensional codes, and obtaining the key areas of interest of the checkerboard corners on the visible light image according to the homography transformation matrix and the preset areas of the checkerboard corners on the standard image of the marker, wherein the preset area is a square area with the corner of the checkerboard as the center and the corners of adjacent areas comprising two-dimensional codes as the diagonal vertices.

6. The target tracking method according to claim 5, wherein according to the key area of interest and the depth image, the method comprises calculating the corresponding checkerboard corners' 3D coordinates as follows:
calculating the 3D coordinates of each pixel in a focus area with the following formula:
(x_{3d}ⁱ,y_{3d}ⁱ,z_{3d}ⁱ)=ƒ(x_{depth}ⁱ,y_{depth}ⁱ,x_{2d}ⁱ,y_{2d}ⁱ),
where i∈(1, . . . , N) indicates i-th pixel among N pixels in the key region of interest, (x_{3d}ⁱ, y_{3d}ⁱ, z_{3d}ⁱ) is the 3D coordinate of i-th pixel, (x_{depth}ⁱ, y_{depth}ⁱ) is the 2D coordinate of i-th pixel in the depth image, (x_{2d}ⁱ, y_{2d}ⁱ) is the 2D coordinate of i-th pixel in the visible light image, ƒ is decided by those parameters of the depth image camera and the visible light camera applied.
calculating the 3D coordinate of corners of the checkerboard with the following formula:
$$( {x}_{3 d}^{c} , {y}_{3 d}^{c} , {z}_{3 d}^{c} ) = \frac{1}{N} {\sum }_{1}^{N} ( {x}_{3 d}^{i} , {y}_{3 d}^{i} , {z}_{3 d}^{i} ) ,$$
where (x_{3d}^{c}, y_{3d}^{c}, z_{3d}^{c}) denotes the 3D coordinates of the checkerboard's corner.

7. The target tracking method according to claim 5, wherein according to the key area of interest and the depth image, the 3D coordinates of corresponding checkerboard corners that have angles and/or deformation on the depth image are calculated as follows:
calculating 3D coordinates of four corners of the area of focus with the following formula:
(x_{3d}ⁱ,y_{3d}ⁱ,z_{3d}ⁱ)=ƒ(x_{depth}ⁱ,y_{depth}ⁱ,x_{2d}ⁱ,y_{2d}ⁱ),
where i∈(1, . . . , 4) indicates the four corners of the area of focus, (x_{3d}ⁱ, y_{3d}ⁱ, z_{3d}ⁱ) is the 3D coordinate of i-th area corner, (x_{depth}ⁱ, y_{depth}ⁱ) denotes the 2D coordinate of i-th area corner in the depth image, (x_{2d}ⁱ, y_{2d}ⁱ) denotes the 2D coordinate of i-th area corner in the visible light image, ƒ is decided by those parameters of the depth image camera and the visible light camera applied.
calculating a center point's coordinate by the interpolation of the 3D coordinates of four area corners:
$$( {x}_{3 d}^{c} , {y}_{3 d}^{c} ) = \frac{1}{4} {\sum }_{1}^{4} ( {x}_{3 d}^{i} , {y}_{3 d}^{i} ) .$$
By fitting plane P: k·x+1·y+m·z=0 to make the following formula establish:
(k,l,m)˜argmin Σ₁^{N}(k·x−x_{3d}ⁱ)²+(l·y−y_{3d}ⁱ)²+(m·z−z_{3d}ⁱ)²;
according to x_{3d}^{c}, y_{3d}^{c}, k, l, m and plane formula P, calculating the 3D checkerboard's corner coordinate (x_{3d}^{c}, y_{3d}^{c}, z_{3d}^{c}).

8. The target tracking method according to claim 1, wherein the marker comprises a flexible planar substrate.

9. The target tracking method according to claim 1, wherein the contrasting colors are black and white.

10. The target tracking method according to claim 1, further comprising the step of moving a robotic arm of the surgical robot.

11. The target tracking method according to claim 1, further comprising the step of moving a surgical tool of the surgical robot.

12. The target tracking method according to claim 11, wherein the surgical tool is selected from the group consisting of a drill guide, a drill, a puncture needle, scissors, a grasper, and a needle holder.

13. The target tracking method according to claim 1, further comprising the step of performing a surgical operation with the surgical robot.

14. The target tracking method according to claim 1, wherein the surgical operation is selected from the group consisting of performing a drilling operation, performing a cutting operation, and performing a grasping operation.

15. A robot, wherein the robot comprises:
a visible light image acquisition module for acquiring a visible light image of a marker attached to the surface of the tracked target, wherein the marker is provided with a checkerboard pattern comprising adjacent areas of contrasting color, and a two-dimensional code is provided inside square-shaped areas of the checkerboard;
a depth image acquisition module for acquiring the depth image of the marker;
an image processing module for detecting the two-dimensional code on the visible light image, obtaining the corner point's two-dimensional (2D) coordinates and ID the two-dimensional code on the marker, and according to the depth image, the 2D coordinates of two-dimensional code's corner point and the two-dimensional code's ID, obtaining the checkerboard corner points' three-dimensional (3D) coordinates on the marker, and obtaining the position information of the tracked target in 3D space according to the checkerboard corner points' 3D coordinates, wherein the location information is used to track the tracked target; and
an execution module for generating a motion instruction to the robot according to the continuously obtained position information of the tracked target in the 3D space, and controlling the robot to follow the movement of the tracked target in the 3D space.

16. A target tracking system, wherein the system comprises:
a marker attached to a surface of a tracked object, wherein the marker is provided with a black and white checkerboard pattern, and two-dimensional codes are arranged inside square-shaped areas of the checkerboard; and
the robot of claim 15.
